(19) United States
(12) Patent Application Publication
(10) Pub. No.: US 2021/0244443 A1
(43) Pub. Date: Aug. 12, 2021

(54) BONE DISPLACEMENT SYSTEM AND METHOD

(71) Applicant: NEXTREMITY SOLUTIONS, INC., Warsaw, IN (US)

(72) Inventors: Matthew COYNE, Warsaw, IN (US); Gregory DENHAM, Warsaw, IN (US)

(73) Assignee: NEXTREMITY SOLUTIONS, INC., Warsaw, IN (US)

(21) Appl. No.: 17/172,672

(22) Filed: Feb. 10, 2021

Related U.S. Application Data
(60) Provisional application No. 62/972,369, filed on Feb. 10, 2020.

(51) Int. Cl. A61B 17/66; A61F 2/46

(52) U.S. Cl. CPC A61B 17/66; A61B 17/1739; A61F 2/4606

(57) ABSTRACT

A bone displacement system includes a body having a first arm and a second arm. The first arm is coupled to a first bone contacting leg configured to be connected to a first bone portion. The second arm is coupled to a second bone contacting leg configured to be connected to a second bone portion. A force application fixture is coupled to the first arm and the first bone contacting leg and configured to apply a force between the first arm and the first bone contacting leg.

### CROSS-REFERENCE TO RELATED APPLICATIONS

The present application claims priority to U.S. Provisional Application No. 62/972,369 filed on Feb. 10, 2020 (Attorney Docket No. 3768.092P1), which is incorporated herein by reference in its entirety.

### BACKGROUND OF THE INVENTION

The present invention generally relates to osteotomy compression or distraction, displacement, and fixation.

### BACKGROUND INFORMATION

During an osteotomy surgical procedure, bone displacement is manually performed. Current distraction techniques limit the amount of bone preparation possible and existing compression techniques often provide for an inadequate fit. Current fixation techniques involve wiring to support and maintain bone position prior to fixation, often leading to inadequate or misaligned fixation.

Thus, a need exists for better controlled displacement, distraction, compression, and fixation during osteotomy surgical procedures.

### SUMMARY OF THE INVENTION

The present invention provides, in one aspect, a bone displacement system which includes a body having a first arm and a second arm. The first arm is coupled to a first bone contacting leg configured to be connected to a first bone portion. The second arm is coupled to a second bone contacting leg configured to be connected to a second bone portion. A force application fixture is coupled to the first arm and the first bone contacting leg and configured to apply a force between the first arm and the first bone contacting leg. These, and other objects, features and advantages of this invention will become apparent from the following detailed description of the various aspects of the invention taken in conjunction with the accompanying drawings.

### BRIEF DESCRIPTION OF THE DRAWINGS

FIG. 1 is a top perspective view of a positioning template, in accordance with an aspect of the present invention;

FIG. 2 is a top perspective view of guide wires and a drill positioned using the positioning template in FIG. 1, in accordance with an aspect of the present invention;

FIG. 3 is a perspective view of an implant installer and attached implant engaged with the guide wires of FIG. 2, in accordance with an aspect of the present invention;

FIG. 4 is a top perspective view of the implant installer and attached implant of FIG. 3, in accordance with an aspect of the present invention;

FIG. 5 is a top perspective view of the attached implant of FIG. 3, in accordance with an aspect of the present invention;

FIG. 6 is a perspective view of a shift jig connected to the implant of FIG. 3, in accordance with an aspect of the present invention;

FIG. 7 is a top perspective view of the shift jig of FIG. 6, in accordance with an aspect of the present invention;

FIG. 8 is a top perspective view of the shift jig of FIG. 6, in accordance with an aspect of the present invention;

FIG. 9 is a side perspective view of the shift jig of FIG. 6, in accordance with an aspect of the present invention;

FIG. 10 is a perspective view of a drill guide in a first position connected to the shift jig of FIG. 6, in accordance with an aspect of the present invention;

FIG. 11 is a perspective view of a coupler of the shift jig connected to the implant of FIG. 6, in accordance with an aspect of the present invention;

FIG. 12 is a top perspective view of the drill guide in a second position connected to the shift jig of FIG. 6, in accordance with an aspect of the present invention;

FIG. 13 is a top perspective view of the shift jig of FIG. 6, in accordance with an aspect of the present invention;

FIG. 14 is a top perspective view of the implant of FIG. 3 connected to two bone segments, in accordance with an aspect of the present invention;

FIG. 15 is a side perspective view of the implant connected to the two bone segments of FIG. 14, in accordance with an aspect of the present invention;

FIG. 16 is a cut-away superior perspective view of the implant connected to the two bone segments of FIG. 14, in accordance with an aspect of the present invention;

FIG. 17 is a cut-away inferior perspective view of the implant connected to the two bone segments of FIG. 14, in accordance with an aspect of the present invention;

FIG. 18 is a cut away lateral perspective view of the implant connected to the two bone segments of FIG. 14, in accordance with an aspect of the present invention;

FIG. 19 is a perspective view of a post fastener, in accordance with an aspect of the present invention;

FIG. 20 is a perspective view of the post fastener of FIG. 19 connected to the implant and the implant installer of FIG. 3, in accordance with an aspect of the present invention;

FIG. 21 is a perspective view of the post fastener of FIG. 19 engaged with a shift jig, in accordance with an aspect of the present invention;

FIG. 22 is a perspective view of an implant attachable to the implant installer of FIG. 3 and engageable with the guide wire of FIG. 2, in accordance with an aspect of the present invention;

FIG. 23 is a perspective view of an implant attachable to the implant installer of FIG. 3 and engageable with the guide wire of FIG. 2, in accordance with an aspect of the present invention;

FIG. 24 is a perspective view of an implant attachable to the implant installer of FIG. 3 and engageable with the guide wire of FIG. 2, in accordance with an aspect of the present invention;

FIG. 25 is a perspective view of an implant attachable to the implant installer of FIG. 3 and engageable with the guide wire of FIG. 2, in accordance with an aspect of the present invention;

FIG. 26 is a perspective view of an implant attachable to the implant installer of FIG. 3 and engageable with the guide wire of FIG. 2, in accordance with an aspect of the present invention;

FIG. 27 is a perspective view of a shift jig engaged with a first metatarsal bone, in accordance with an aspect of the present invention;

FIG. 28 is a side perspective view of the shift jig of FIG. 27, in accordance with an aspect of the present invention;

FIG. 29 is a superior perspective view of the shift jig of FIG. 27, in accordance with an aspect of the present invention;

FIG. 30 is a front perspective view of the shift jig of FIG. 27, in accordance with an aspect of the present invention;

FIG. 31 is a superior perspective view of a coupler in a second position connected to the shift jig of FIG. 27, in accordance with an aspect of the present invention;

FIG. 32 is a front perspective view of the coupler of FIG. 31 in the second position connected to the shift jig of FIG. 27, in accordance with an aspect of the present invention;

FIG. 33 is a superior perspective view of the shift jig of FIG. 27, in accordance with an aspect of the present invention;

FIG. 34 is a superior perspective view of the shift jig of FIG. 27, in accordance with an aspect of the present invention;

FIG. 35 is a cut-away side perspective view of screws inserted into the first metatarsal bone of FIG. 27, in accordance with an aspect of the present invention; and

FIG. 36 is a cut-away side perspective view of screws inserted into the first metatarsal bone of FIG. 27, in accordance with an aspect of the present invention.

### DETAILED DESCRIPTION OF THE INVENTION

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

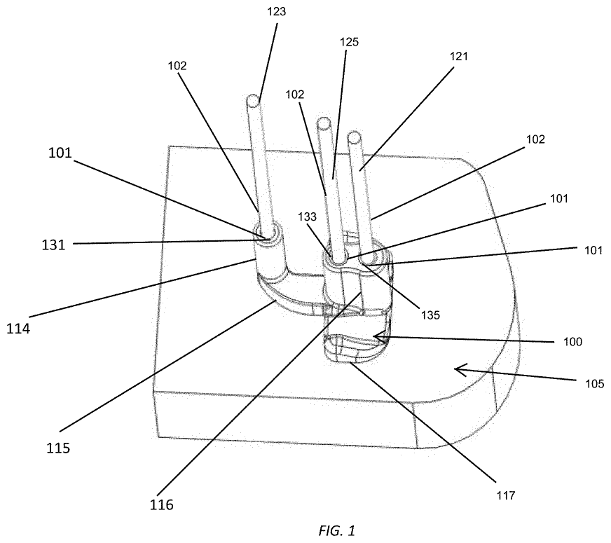

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

The following description references systems, methods, and apparatuses for use in compression/distraction, displacement, and fixation of a calcaneal osteotomy and/or a distal bunion osteotomy. A calcaneal osteotomy involves the cutting and repositioning of a portion of a calcaneus to correct alignment. A distal bunion osteotomy involves the cutting and repositioning of a portion of a first metatarsal bone to correct alignment. However, those possessing an ordinary level of skill in the relevant art will appreciate that other bone osteotomies are suitable for use with the foregoing systems, methods, and apparatuses. Likewise, the various figures, steps, procedures, and work-flows are presented only as an example and in no way limit the systems, methods or apparatuses described to performing their respective tasks or outcomes in different time-frames or orders. The teachings of the present invention may be applied to compression/distraction, displacement, and fixation related to any osteotomy.

Furthermore, there is no intention to be bound by any expressed or implied theory presented in the preceding technical field, background, brief summary, or the following detailed description. It is also to be understood that the specific devices and processes illustrated in the attached drawings, and described in the following specification, are simply exemplary embodiments of the inventive concepts. Hence, specific dimensions and other physical characteristics relating to the embodiments disclosed herein are not to be considered as limiting, unless expressly stated otherwise.

The various embodiments described herein provide for systems, apparatuses, devices, and methods for compression/distraction, displacement, and fixation of a calcaneal osteotomy and/or a distal bunion osteotomy. The various figures and description may refer to a calcaneal osteotomy and/or a distal bunion osteotomy. However, one having ordinary skill in the art will understand that that the following systems, apparatuses, devices, and methods for compression/distraction, displacement, and fixation of a calcaneal osteotomy and/or a distal bunion osteotomy may be used with other bone osteotomies.

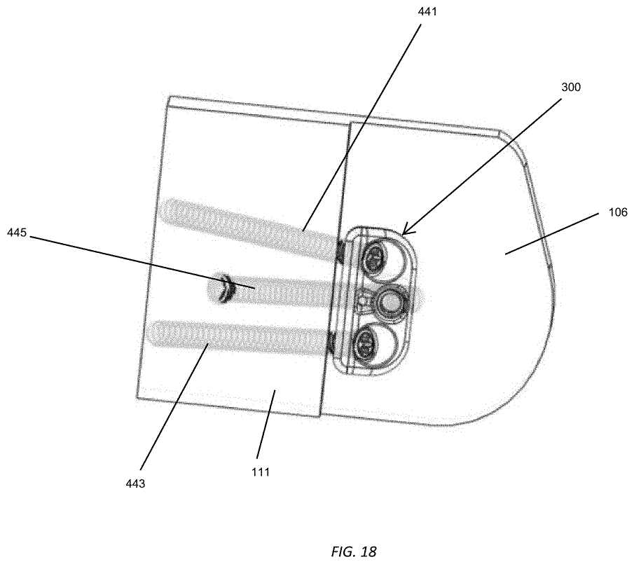

Referring to the drawings, wherein like reference numerals are used to indicate like or analogous components throughout the several views, and with particular reference to FIGS. 1-26 a calcaneal osteotomy system is depicted in various embodiments.

With reference to FIG. 1, a trial or template 100 is depicted seated on a lateral side of a calcaneus 105. Template 100 may have, for example, a body 117, an arm 115 extending from body 117, a first wire support 114 extending out from arm 115 and away from calcaneus 105, and a second wire support 116 extending out from body 117 and away from calcaneus 105. Template 100 may have, for example, wire holes 101. Wire holes 101 may include a first wire hole 131 located on first wire support 114, and a second wire hole 133 and a third wire hole 135 located on second wire support 116 as depicted in FIG. 1. Template 100 is depicted with three wire holes 101, however the number of wire holes 101 may vary with, for example, bone size, bone density, and/or surgical desire. First wire hole 131 extends through first wire support 114 and arm 115. First wire support 114 may be a conical or a tubular structure; however, the shape of first wire support 114 may, for example, be any shape and/or first wire support 114 may be configured (e.g., shaped and dimensioned) to have multiple wire holes 101. Second wire hole 133 and a third wire hole 135 extend through second wire support 116 and through body 117. Second wire support 116 may be a conical or a tubular structure; however, the shape of second wire support 116 may, for example, be any shape and/or second wire support 116 may be configured (e.g., shaped and dimensioned) to have one instance or multiple instances of wire holes 101.

Wire holes 101 may be, for example, used as drill guides or as supports for a drill guide (not shown). Each instance of wire holes 101 may be used, to drill a hole (not shown) into calcaneus 105. Wire holes 101 are depicted with guide wires or k-wires 102 inserted, however the number of instances of wire holes 101 with guide wires 102 inserted may vary with, for example, bone size, bone density, and/or surgical desire. Wire holes 101 are depicted as circular, however wire holes 101 may be any shape configured (e.g., shaped and dimensioned) to accommodate guide wires 102. Guide wires 102 may be, for example, inserted into holes drilled into calcaneus 105. Template 100 is used to determine a position for an implant and to insert guide wires 102 to guide or position instruments during a calcaneal osteotomy surgical procedure. As depicted in FIG. 1, guide wires 102 include a first guide wire 121 positioned towards a posterior of calcaneus 105, a second guide wire 123 positioned towards an anterior of calcaneus 105, and a third guide wire 125 positioned between first guide wire 121 and second guide wire 123. As a calcaneal osteotomy surgical procedure involves cutting a portion of calcaneus 105, wire holes 101 with inserted guide wires 102 may, for example, be positioned on opposing sides of a prospective calcaneal cut.

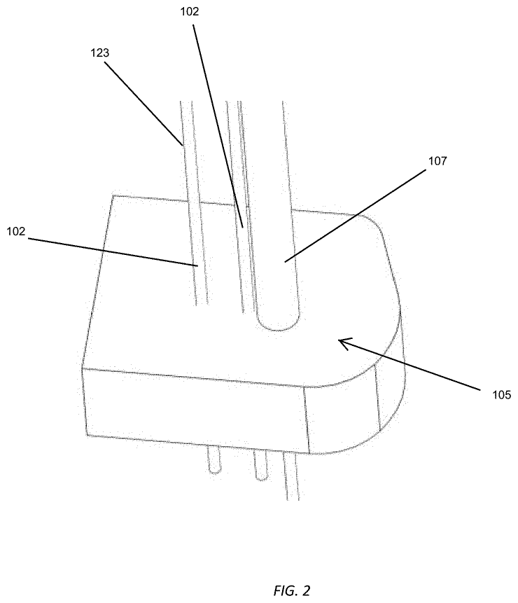

After the desired number of guide wires 102 are inserted into calcaneus 105 via wire holes 101, template 100 may be, for example, removed. A drill 107 may be inserted over first guide wire 121, with first guide wire 121 being used as a guide for drill 107 to drill a hole for an implant, as depicted in FIG. 2, for example.

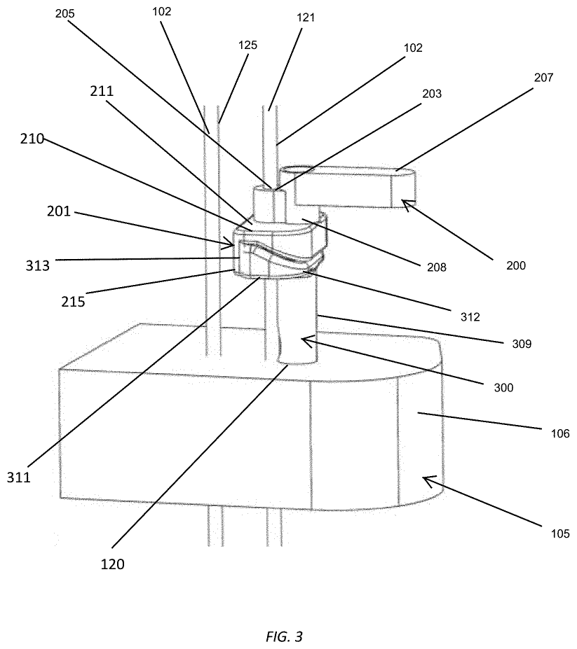

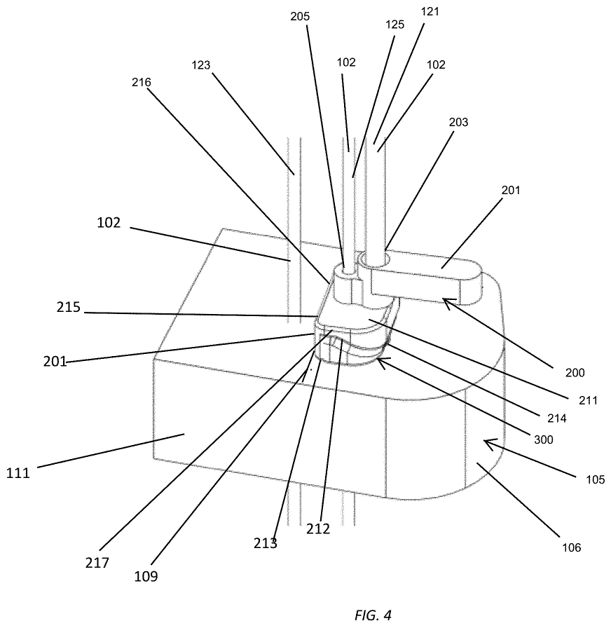

An implant 300 may be removably coupled to an installer 200, as depicted in FIGS. 3 and 4. Installer 200 may include a body 210 between a top surface 211 and a bottom surface 212, a first side 215 and a second side 214, and a first end 216 and a second end 217. A first tube 208 may extend from top surface 211 of body 210 and in a direction away from calcaneus 105 towards a handle 207. A second tube 209 may extend from top surface 211 of body 210 and in a direction away from calcaneus 105. First tube 208 and second tube 209 may be, for example, approximately perpendicular to top surface 211 and adjacent to each other. Handle 207 extends away from body 210 and may, for example, extend from first tube 208 in an approximately perpendicular direction to a longitudinal dimension of second side 214. First tube 208 may have, for example, a first hole 203 and second tube 209 may have, for example, a second hole 205. First hole 203 may, for example, extend through first tube 208 and through body 210. Second hole 205 may, for example, extend through second tube 209 and through body 210. First tube 208 may be closer to second side 214 and the posterior of calcaneus 105 and second tube 209 may be closer to first side 215 and the anterior of calcaneus 105. First side 215 may, for example, extend towards calcaneus 105 in an approximately perpendicular direction to top surface 211. First side 215 may have, for example, a cut face 201 and an inner side 213. Body 210 may have, for example, a bottom surface 212 that angles away from top surface 211, forming a wedge shape extending from inner side 213 of first side 215 towards second side 214, with second side 214 being a thicker side of the wedge shape.

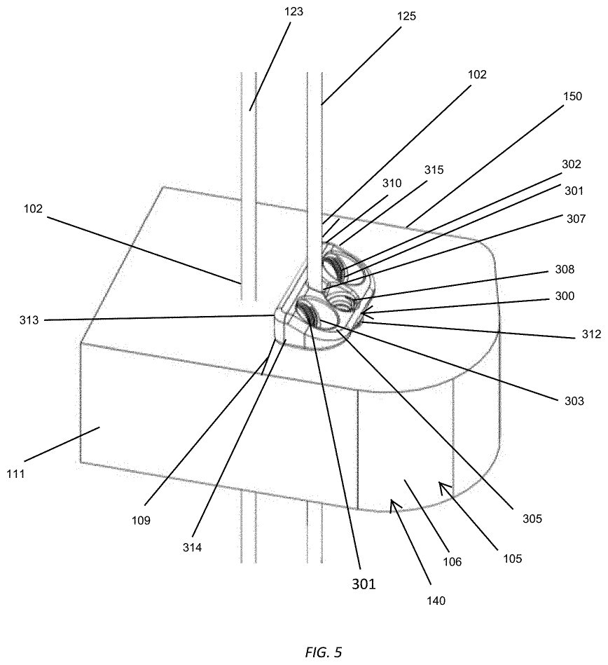

Implant 300 may include a body 310 between a top surface 305 and a bottom surface 311, a first end 314 and a second end 315, and a first side 312 and a second side 313, as depicted in FIGS. 5 and 25, for example. Body 310 further has a post 309 extending from bottom surface 311 of body 310, for insertion into calcaneus 105, as depicted in FIG. 3, for example. Post 309 may, for example, be aligned approximately perpendicular to a longitudinal dimension of bottom surface 311. As depicted in FIG. 3, post 309 is inserted into calcaneus 105. As depicted in FIG. 5, top surface 305 is angled in the direction of bottom surface 311 between second side 313 and first side 312, with body 310 forming a wedge shape between second side 313 and first side 312, and second side 313 forming a thicker end of the wedge shape. First end 314 and second end 315 may be, for example, triangular. A first wire hole 307, positioned towards second side 313, extends through body 310 from top surface 305 to bottom surface 311. A second wire hole 308, positioned towards first side 312, extends through body 310 from top surface 305 through bottom surface 311 and through post 309. Implant 300 further has screw holes 301, extending through body 310 from top surface 305 to bottom surface 311. Screw holes 301 may include, for example, a first screw hole 302 positioned closer to first end 314 and a second screw hole 303 positioned closer to second end 315. First end 314 may be positioned closer to a plantar side 140 of calcaneus 105 and second end 315 may be positioned close to a dorsal side 150 of calcaneus 105.

With reference to FIGS. 3-5, implant 300 may be removably coupled to installer 200. For example, implant 300 may be placed onto or in contact with installer 200. Top surface 305 of implant 300, may be adjacent to bottom surface 212 of installer 200, with second side 313 of implant 300 adjacent to inner surface 213 of installer 200. Implant 300 and installer 200 may be positioned on first guide wire 121 and third guide wire 125, with first guide wire 121 and third guide wire 125 used to guide implant 300 and installer 200 into position, i.e. by being received in holes (307 and 308) of implant 300 and holes (203 and 205) of installer 200. First guide wire 121 may pass through first guide wire hole 203 and second wire hole 308. Third guide wire 125 may pass through second guide wire hole 205 and first wire hole 307.

Post 309 is depicted as partially inserted into drill hole 120 created by drill 107 (FIG. 2) in FIG. 3 and fully inserted into drill hole 120 in FIG. 4, seating implant 300 on calcaneus 105. The connected combination of implant 300 and installer 200 may be used to, for example, provide a reference surface for a surgical cut of calcaneus 105. First side 215 has a cut face 201 which may be, for example, positioned parallel to a prospective cut. A surgical cut 109, may be, for example, made using cut face 201 to separate a posterior calcaneal section 106 from an anterior calcaneal section 111 of calcaneus 105. First guide wire 121 and third guide wire 125 may, for example, remain in posterior calcaneal section 106 or first guide wire 121, may be, for example, removed as is surgically desired. Second guide wire 123 may, for example, remain in anterior calcaneal section 111, as is surgically desired.

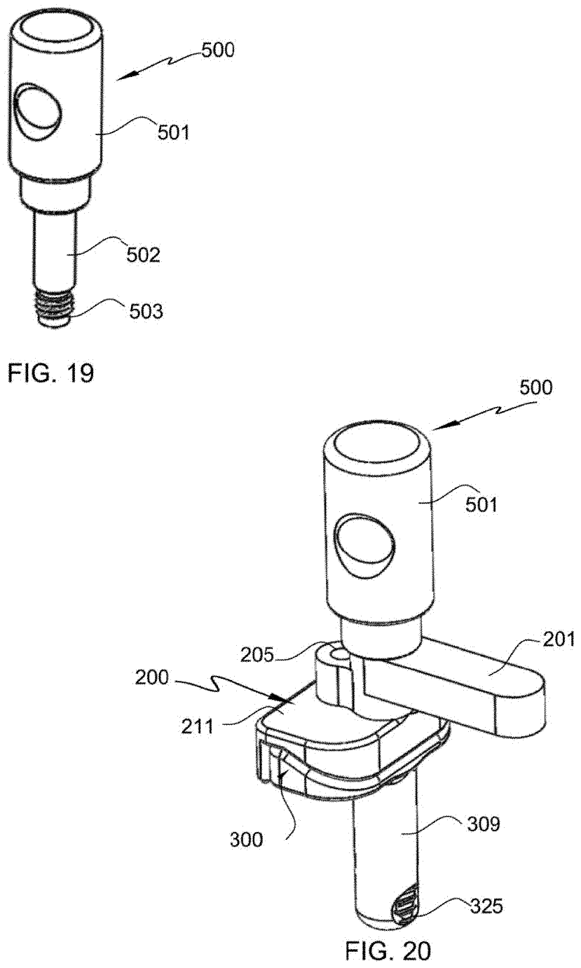

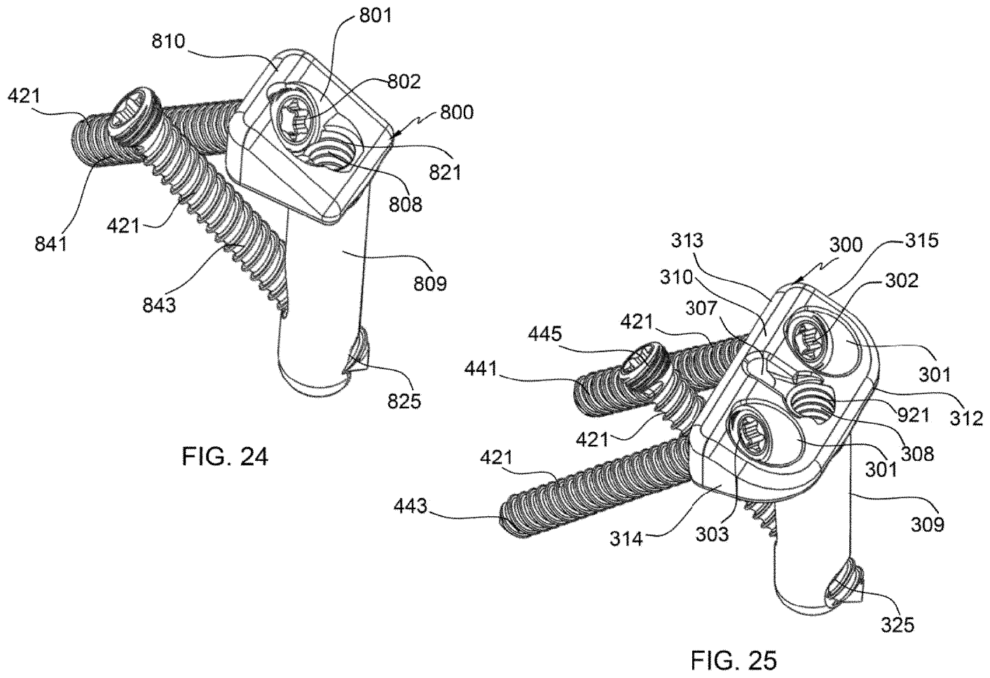

With reference to FIGS. 19-20, a post fastener 500 may be used to connect implant installer 200 to implant 300. With reference to FIG. 19, post fastener 500 may be, for example, cylindrical with a head section 501 and a fastening section 502 extending therefrom. Fastening section 502 may have, for example, a threaded section 503 extending longitudinally along at least a portion of fastening section 502. For example, second wire hole 308 may have a smooth or threaded enclosure wall 321 through at least a portion of the interior of post 309, as depicted in FIGS. 24-26. With reference to FIG. 20, post fastener 500 may be inserted through first wire guide hole 203 (as depicted in FIGS. 3 and 4) and threaded section 503 may be engaged with the enclosure wall 321 of second wire hole 308 of post 309, removably coupling implant installer 200 and implant 300. Implant installer 200 and implant 300 may then be, for example, placed onto guide wire 125 and more specifically, second guide wire hole 205 and guide hole 307 may be aligned for placement onto guide wire 125 and insertion of post 309 onto posterior calcaneal section 106, as depicted in FIGS. 3 and 4.

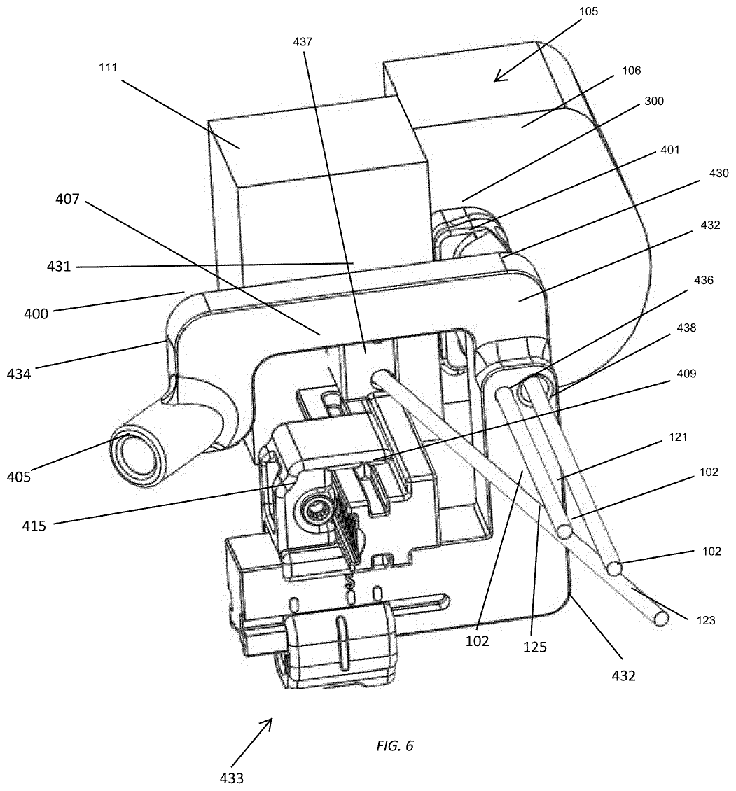

With reference to FIGS. 6-8, a bone adjustment mechanism or shift jig 400 may be placed onto first guide wire 121 received in a first hole 438, second guide wire 123 received in a second hole 437, and third guide wire 125 received in a third hole 436, with a coupler 401 placed onto and/or connected to top surface 305 of implant 300. Coupler 401 engaged with implant 300 may be considered a second bone engagement leg. Shift jig 400 may have, for example, a body 430 formed of a first segment 431 approximately perpendicular to a second segment 432, a third segment 433 approximately perpendicular to second segment 432, a fourth segment 434 approximately perpendicular to first segment 431 and an open interior or cavity between the four segments or arms. Fourth segment 434 may, for example, extend from first segment 431 towards third segment 433 but does not contact third segment 433, leaving an opening. Fourth segment 434 may have, for example, a drill guide 405 extending away from fourth segment. Drill guide 405 may, for example, have a hole extending though drill guide 405 and fourth segment 434, forming a cylindrical tube. Shift jig 400 may include force application fixtures, such as, a compression-distraction fixture 403 and a displacement fixture 409 movably coupled with and on opposing sides of third segment 433. Displacement fixture 409 may be movably coupled to a guide segment 407. Coupler 401 may extend away from second segment 432 in an approximately perpendicular direction relative to a longitudinal dimension of second segment 432 and towards calcaneus 105. First hole 438 and third hole 436 extend through second segment 432 and through coupler 401, with first guide wire 121 passing through first hole 438 and third guide wire 125 passing through third hole 436. Second hole 437 extends through guide segment 407 with second guide wire 123 passing through, with guide segment 407 positioned on section 111. First hole 438, second hole 437, and third hole 436 are aligned to position shift jig 400 onto guide wires 102.

While first segment 431, second segment 432, third segment 433, and fourth segment 434 of body 430 of shift jig 400 are depicted, other embodiments of a shift jig may, for example, have configurations with more segments or less segments, or arms, and such segments may, for example, have configurations forming a body (e.g., body 430) of a shift jig (e.g., shift jig 400) with a different shaped configuration. While shift jig 400 is depicted as having segments arranged in a square shape, other segment configurations may be used to form shift jig and body thereof having different shapes.

With reference to FIGS. 6-8, compression-distraction fixture 403 has a housing 406 to hold a first power screw 411. Third segment 433 may, for example, have a rack 417 along the length of on an exterior side, with compression-distraction fixture 403 engaged with rack 417 and with first power screw 411 acting as a worm gear movably coupled with rack 417. Compression-distraction fixture 403 may be, for example, movable along rack 417 by actuating first power screw 411 manipulated by a screwdriver 413.

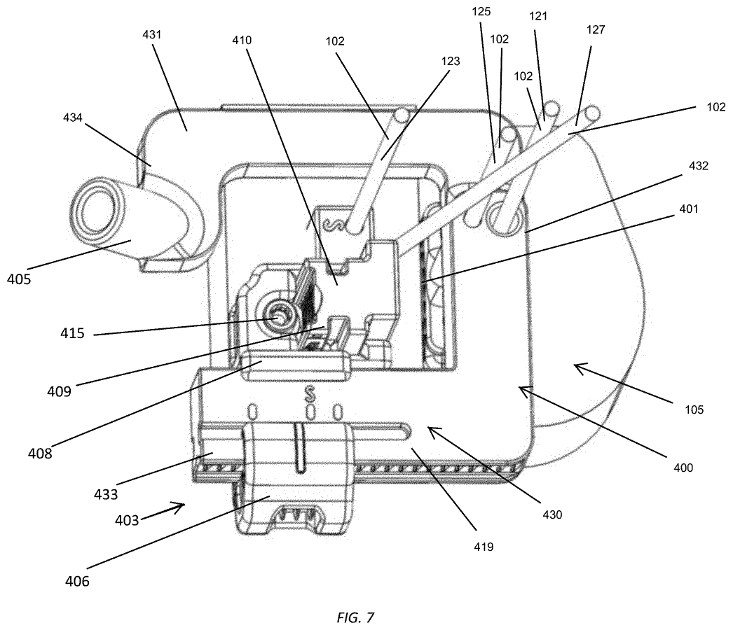

With reference to FIGS. 7-9, a fourth guide wire 127 may be placed through a fourth hole 439 in guide segment 407 and into anterior calcaneal section 111 at, for example, a divergent angle relative to second guide wire 123 to lock the position of guide segment 407 relative to anterior calcaneal section 111. Guide segment 407 may be coupled to a threaded rack post 410, with guide segment 407 and/or rack post 410 seated on anterior calcaneal section 111. Guide segment 407 and rack post 410 may be together considered a first bone engagement leg. Displacement fixture 409 has housing 408 to hold a second power screw 415. Displacement fixture 409 may be, for example, coupled to third segment 433 and located on a side of third segment 433 opposite compression-distraction fixture 403. Housing 408 for displacement fixture 409 may be, for example, engaged with rack post 410 and with second power screw 415 acting as a worm gear movably coupled with rack post 410. Displacement fixture 409 may be, for example, movable along rack post 410 by actuating second power screw 415 manipulated by a screwdriver (e.g., screwdriver 413) and thereby moving shift jig 400 along rack post 410.

Manipulating first power screw 411 may, for example, move posterior calcaneal section 106 in an approximately anterior/posterior direction providing compression or distraction. Manipulating power screw 415 may, for example, move shift jig 400 and posterior calcaneal section 106 in an approximately medial/lateral direction providing for displacement of posterior calcaneal section 106 relative to calcaneus section 111. Both compression-distraction fixture 403 and displacement fixture 409 may, for example, maintain or even lock their position when a screwdriver (e.g., screwdriver 413) is not engaged with first power screw 411 or second power screw 415, respectively.

After a desired placement of posterior calcaneal section 106 is achieved relative to anterior calcaneal section 111, shift jig 400 may hold posterior calcaneal section 106 in place for fixation, for example.

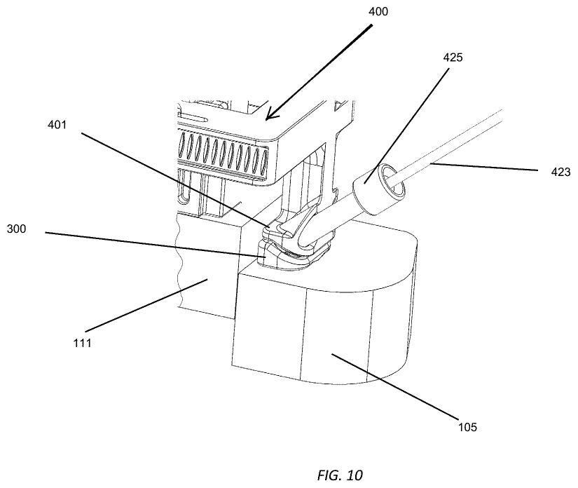

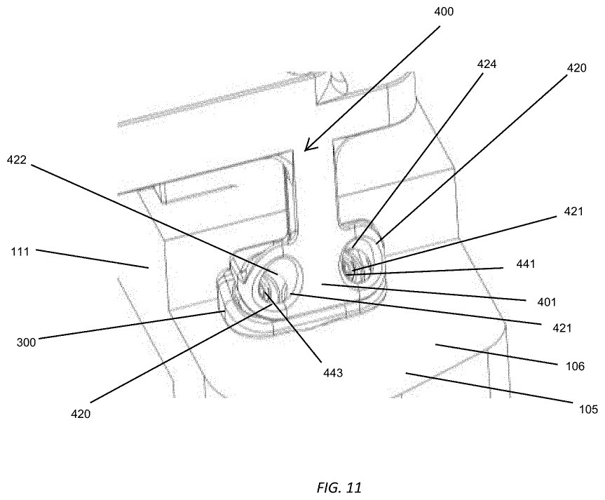

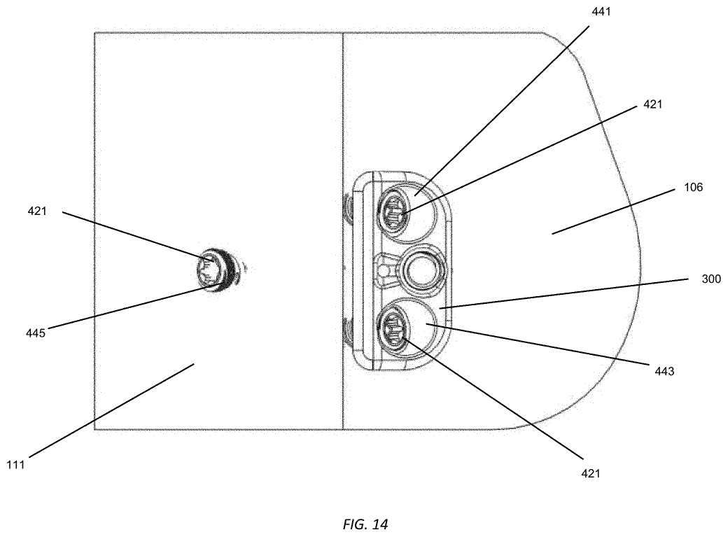

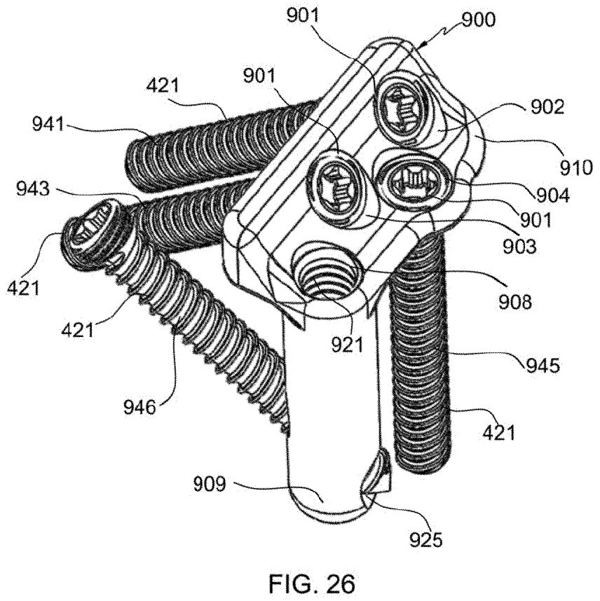

With reference to FIGS. 5 and 9-11, coupler 401 may have screw guides 420 that correspond to screw holes 301. Screw guides 420 may have, for example, a first screw guide 424 and a second screw guide 422. First screw guide 424 and second screw guide 422 may, for example, align with first screw hole 302 and second screw hole 303, respectively. A drill tube 425 may be, for example, inserted into first screw guide 424 and used to guide a drill bit 423 for drilling into posterior calcaneal section 106 and anterior calcaneal section 111 in preparation for bone screws 421. Drill tube 425 may be, for example, removed from first screw guide 424 and inserted into second screw guide 422 to guide a drill bit 423 for drilling into posterior calcaneal section 106 and anterior calcaneal section 111 in preparation for bone screws 421. The order in which the holes are drilled may be reversed and may be determined by surgical preference. Drill tube 425 may, for example, have a cylindrical shape. Bone screws 421 may, for example, include a first bone screw 441, a second bone screw 443, and a third bone screw 445, as depicted in FIGS. 14 and 25. Drill tube 425 may, for example, be used to guide bone screws 421 or may be removed prior to inserting bone screws 421, as depicted in FIG. 10. After bone preparation, first bone screw 441 may be inserted through first screw guide 424 into first screw hole 302 (FIG. 5) and second bone screw 443 may be inserted through second screw guide 422 into second screw hole 303 (FIG. 5), connecting posterior calcaneal section 106 to anterior calcaneal section 111, as depicted in FIG. 11. A fourth bone screw may be utilized with an implant 700 (e.g., a fourth bone screw 746) and with an implant 900 (e.g., a fourth bone screw 946), as depicted in FIGS. 23 and 26.

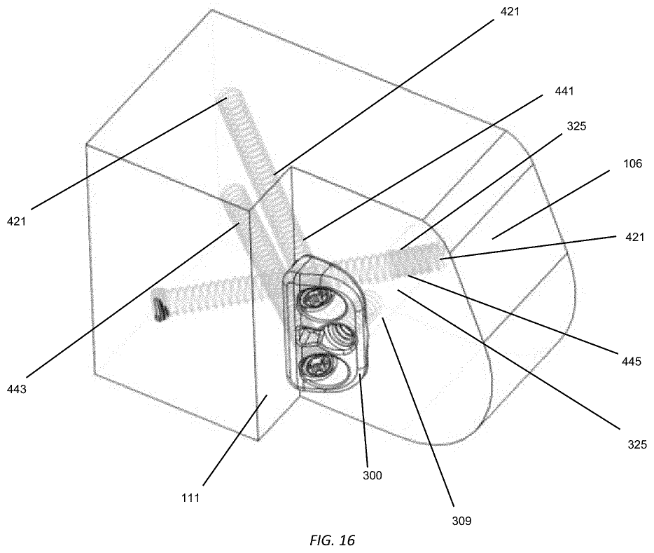

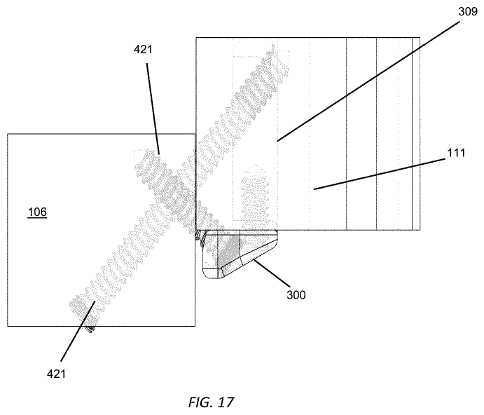

Post 309 may have, for example, a post hole 325 angled and transversely passing through post 309 as depicted in FIGS. 16 and 17. Post hole 325 may have threading or screw thread catching protrusions therethrough.

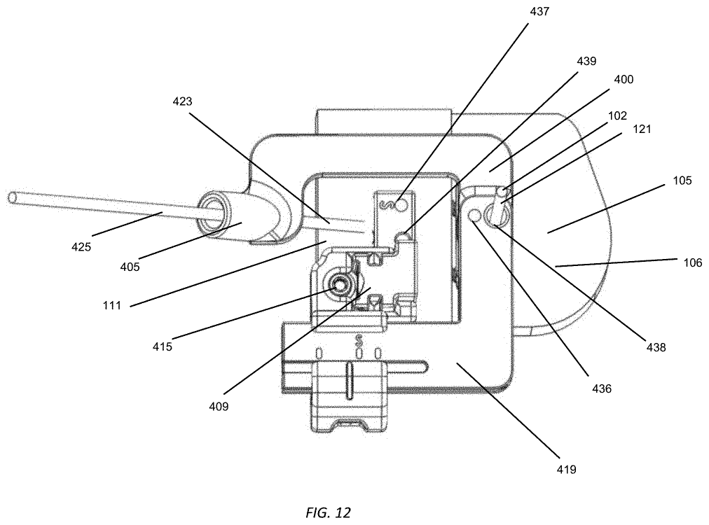

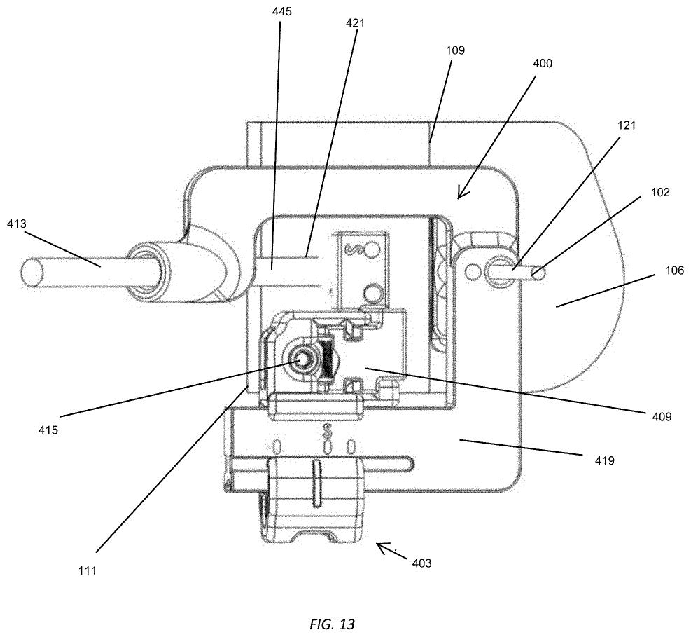

With reference to FIGS. 12 and 13, drill tube 425 may be, for example, configured (e.g., shaped and dimensioned) for insertion into drill guide 405 and used to guide a drill bit 423 for drilling into and through anterior calcaneal section 111 and posterior calcaneal section 106 in preparation for third bone screw 445. Drill guide 405 may be, for example, configured to align with a post hole (e.g., post hole 325). A hole created through anterior calcaneal section 111 and posterior calcaneal section 106 in preparation for third bone screw 445 may, for example, end near or meet post hole (e.g. post hole 325). Drill tube 425 may, for example, be used to guide third bone screw 445 or may be removed prior to inserting third bone screw 445.

With reference to FIGS. 13-18, after bone preparation, third bone screw 445 may, for example, pass through anterior calcaneal section 111, through posterior calcaneal section 106 and through post hole 325 to connect with post 309. Post hole 325 may, for example, have threading matching third bone screw 425 to provide for connection with post 309.

With reference to FIGS. 13-18, bone screws 421 may, for example, have an angled cut to a screw head to provide for a flush fitting with a bone surface. However, screws may, for example, be used without an angled cut head.

With reference to FIGS. 6-9, 12, and 13, shift jig 400 is depicted as being attached with guide wires 102. However, screws may be used in place of or in addition to guide wires 102, as surgically desired.

With reference to FIGS. 13-15, guide wires 102 and shift jig 400 may be removed, leaving implant 300 connected to posterior calcaneal section 106 and anterior calcaneal section 111. Shift jig 400 may, for example, hold posterior calcaneal section 106 in a surgically desired position while bone preparation and fixation are taking place.

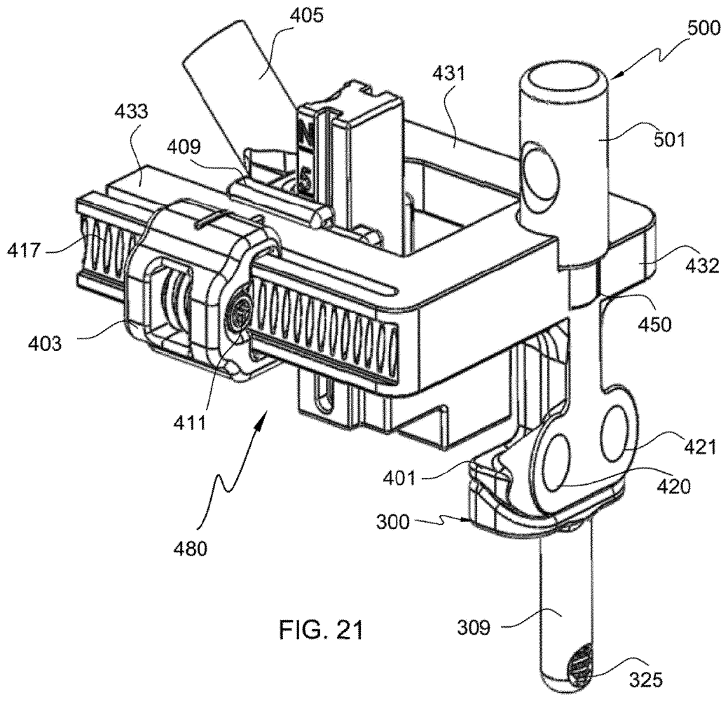

In another example, a shift jig 480 is depicted in FIG. 21. Shift jig 400 has been described in detail above (FIGS. 6-13) and due to the similarity with shift jig 480, differentiating features will only be described for shift jig 480. Shift jig 480 includes a post fastener slot 450 positioned through second segment 432 and through coupler 401. Post fastener slot 450 may be, for example, configured (e.g., shaped and dimensioned) to receive a post fastener 500, with threaded segment 503 removably coupled to implant 300. Post fastener slot 450 may, for example, extend through second segment 432 and through coupler 401 depicted in FIG. 21, in place of first hole 436 and third hole 438 depicted in FIGS. 6-8, 12, and 13. Thus, post fastener 500 may be, for example, used in place of first guide wire 121 and third guide wire 125 depicted in FIGS. 6-8, to fasten shift jig 480 to implant 300. In another example, post fastener slot 450 as depicted in FIG. 21 may be located on or extend through second segment 432 and coupler 401 adjacent to one or both of a first hole 436 and third hole 438 depicted in FIGS. 6-8, 12, and 13. Thus, post fastener 500 may be, for example, used in addition to one or both of first guide wire 121 and third guide wire 125 to fasten shift jig 480 to implant 300.

Shift jig 480 (FIG. 21) may be attached to a bone (e.g., calcaneus 105, posterior calcaneal section 106; and/or anterior calcaneal section 111) using guide wires and/or screws, as surgically desired.

Referring to FIGS. 1-18, while a calcaneal osteotomy procedure is depicted to move posterior calcaneal section 106 on a left foot in a medial direction, the calcaneal osteotomy surgical procedure is equally valid on the right foot and may be used in procedures where a bone in a calcaneal osteotomy surgical procedure is moved medially or laterally.

Referring to FIG. 1, an exemplary surgical technique in accordance with the present invention for performing a calcaneal osteotomy procedure begins with positioning and seating template 100 onto calcaneus 105. Guide wires 102 are inserted through wire holes 101 and into calcaneus 105.

Referring to FIGS. 2 and 3, a drill 107 is inserted over one instance of guide wires 102 as a desired surgical location and a hole is created for post 309 of implant 300, thereby. Drill 107 is removed. Implant 300 is connected to implant installer 200 as described above and both are inserted onto guide wires 102 previously inserted into calcaneus 105, with post 309 being inserted onto the most posterior guide wire or first guide wire 121 and into post hole 120.

Referring to FIGS. 4 and 5, the surgical technique continues by seating implant 300. A cut is made through calcaneus 105, creating posterior calcaneal section 106 and anterior calcaneal section 111. Implant installer 200 is removed while implant 300 remains in posterior calcaneal section 106.

Referring to FIGS. 6 and 7, shift jig 400 is placed onto guide wires 102 previously installed into calcaneus 105 and coupler 401 is placed onto and/or connected to implant 300. Fourth guide wire 127 is inserted through guide segment 407 and into anterior calcaneal section 111 at a divergent angle relative to second guide wire 123. In another example depicted in FIG. 21, shift jig 480 may be placed onto guide wires 102 and onto implant 300. Shift jig 480 may be attached to a bone (e.g., calcaneus 105, posterior calcaneal section 106; and/or anterior calcaneal section 111) using guide wires and/or screws, as surgically desired. Post fastener 500 may be, for example, placed into post fastener slot 450 and rotated to engage threaded section 503 with threading present in enclosure wall 321 surrounding second wire hole 308. Thus, shift jig 480 and post fastener 500, via coupler 401, may be removably coupled to implant 300.

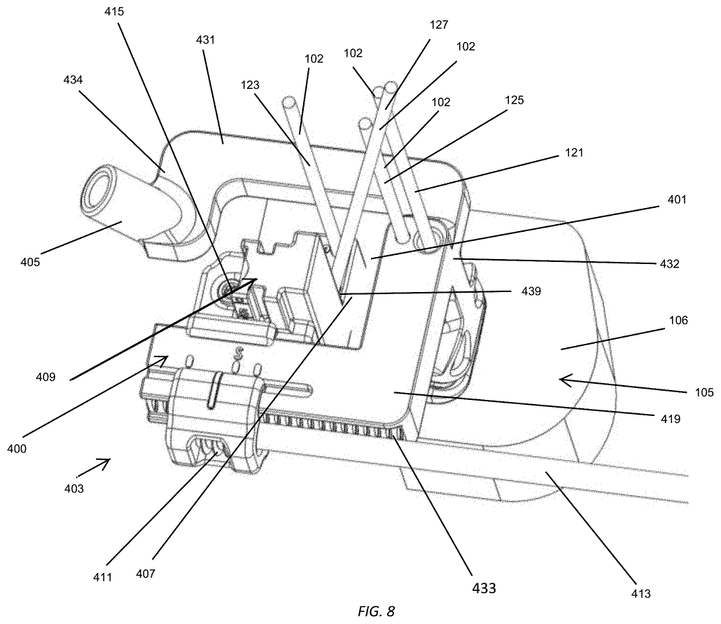

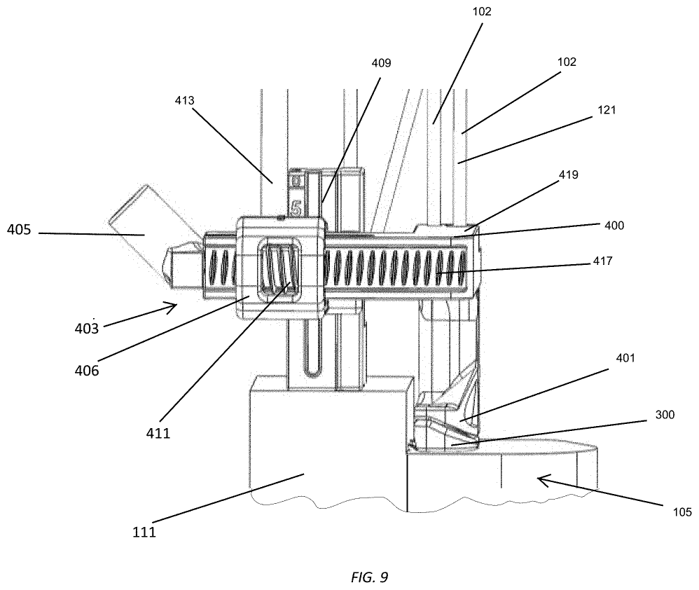

Referring to FIGS. 8 and 9, displacement fixture 409 is used to adjust posterior calcaneal section 106 displacement in the medial-lateral direction relative to anterior calcaneal section 111 of calcaneus 105 and compression-distraction fixture 403 is used to adjust compression or distraction of posterior calcaneal section 106 and anterior calcaneal section 111 in an anterior-posterior direction as described above.

Referring to FIGS. 10 and 11, drill tube 425 is inserted into coupler 401, in particular first screw guide 424 and/or second screw guide 422. Holes are drilled by extending drill bit 423 through drill tube 425 and through posterior calcaneal section 106 into anterior calcaneal section 111 in preparation for inserting bones screws. Drill tube 425 is removed and first bone screw 441 and second bone screw 443 are inserted into implant 300, through posterior calcaneal section 106 and into anterior calcaneal section 111, fastening posterior calcaneal section 106 and anterior calcaneal section 111.

Referring to FIGS. 12 and 13, drill tube 425 is inserted into drill guide 405 and a hole is drilled through anterior calcaneal section 111 and into posterior calcaneal section 106 in preparation for a bone screw. Third bone screw 445 is inserted through anterior calcaneal section 111, through posterior calcaneal section 106, and threaded through post 309, fastening anterior calcaneal section 111 and posterior calcaneal section 106.

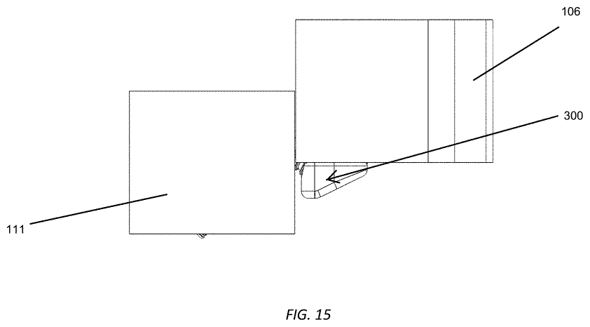

Referring to FIGS. 14 and 15, shift jig 400 and guide wires 102 are removed, leaving implant 300 connected to anterior calcaneal section 111 and posterior calcaneal section 106.

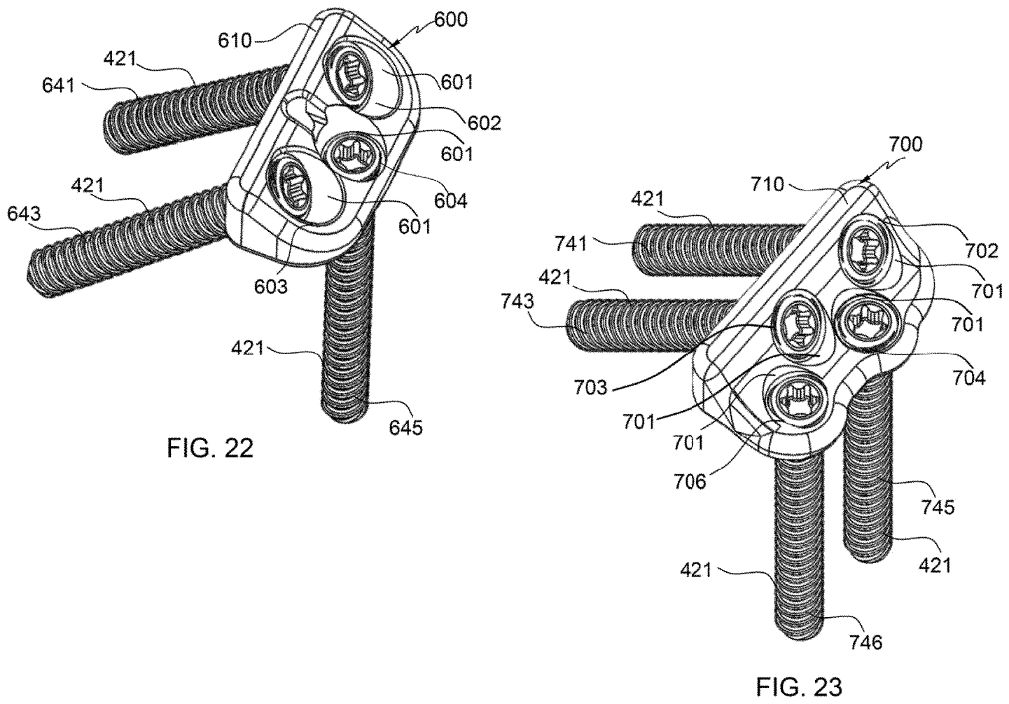

With reference to FIGS. 22-26, bone screw configurations (e.g., a number, location, and alignment of bone screws 421) of implants may vary as is surgically desired. There may be, for example, implant 300, an implant 800, and an implant 900 with a post (e.g. post 309, a post 809, and a post 909) and an implant 600 and an implant 700 without a post. Implant 300, implant 600, implant 700, implant 800, and implant 900 may have, for example, similarly shaped bodies, with body 310, a body 610, a body 710, a body 810, and a body 910 having a wedge shape. However, the various implants (e.g., 300, 600, 700, 800, and 900) have different screw hole configurations and screw hole numbers. The various screw and screw hole configurations and/or post placements may be used to position and anchor posterior calcaneal section 106 to anterior calcaneal section 111 as is surgically desired.

In one example, implant 600 may have three screw holes 601 configured (e.g., shaped and dimensioned) to receive three screws 421 as depicted in FIG. 22, with a first screw 641 inserted through a first hole 602, a second screw 643 inserted through a second hole 603, and a third screw 645 inserted through a third hole 604. First screw 641 and second screw 643 may, for example, be inserted through posterior calcaneal section 106 and into anterior calcaneal section 111. Third screw 645 may be, for example, inserted through third hole 604 and into posterior calcaneal section 106. Thus, implant 600 may have first screw 641 and second screw 643 anchoring implant 600 and posterior calcaneal section 106 to anterior calcaneal section 111, with implant 600 being further anchored into posterior calcaneal section 106 by third screw 645.

In another example, implant 700 may have four screw holes 701 configured (e.g., shaped and dimensioned) for four screws 421 as depicted in FIG. 23, with a first screw 741 inserted through a first hole 702, a second screw 743 inserted through a second hole 703, a third screw 745 inserted through a third hole 704, and a fourth screw 746 inserted through a fourth hole 706. First screw 741 and second screw 743 may be, for example, inserted through posterior calcaneal section 106 and into anterior calcaneal section 111. Third screw 745 and fourth screw 746 may be, for example, inserted into posterior calcaneal section 106. Thus, implant 700 may have, for example, first screw 741 and second screw 743 anchoring implant 700 and posterior calcaneal section 106 to anterior calcaneal section 111, with implant 700 being further anchored into posterior calcaneal section 106 by third screw 745 and fourth screw 746.

There may be, for example, implant 800 having post 809, a post hole 808, and a screw hole 802 configured (e.g., shaped and dimensioned) for a first screw 841 as depicted in FIG. 24, with first screw 841 inserted through screw hole 802. First screw 841 may, for example, be inserted through posterior calcaneal section 106 and into anterior calcaneal section 111. Post 809 may, for example, be inserted into posterior calcaneal section 106. A second screw 443 may, for example, be inserted through anterior calcaneal section 111 and posterior calcaneal section 106 into a post hole 825. Thus, implant 800 may have first screw 841 anchoring implant 800 and posterior calcaneal section 106 to anterior calcaneal section 111 and second screw 843 may, for example, anchor posterior calcaneal section 106, post 809, and implant 800 to anterior calcaneal section 111.

For example, implant 300 may have post 309 and two screw holes 301 configured (e.g., shaped and dimensioned) for two screws 421 as depicted in FIG. 25, with first screw 441 inserted through first hole 302 and second screw 443 inserted through second hole 303. With reference to FIGS. 16-18, and 25, first screw 441 and second screw 443 may, for example, be inserted through posterior calcaneal section 106 and into anterior calcaneal section 111. Post 309 may be inserted into posterior calcaneal section 106. Third screw 445 may be, for example, inserted through anterior calcaneal section 111 and posterior calcaneal section 106 into post hole 325. Thus, implant 300 may have first screw 441 anchoring implant 300 and posterior calcaneal section 106 to anterior calcaneal section 111 and third screw 445 may, for example, anchor posterior calcaneal section 106 and implant 300 to anterior calcaneal section 111.

In another example, implant 900 may include post 909 with a post hole 908 and three screw holes 901 configured (e.g., shaped and dimensioned) for three screws 421 as depicted in FIG. 26, with a first screw 941 inserted through a first hole 902, a second screw 943 inserted through a second hole 903, and a third screw 945 inserted through a third hole 904. First screw 941 and second screw 943 may, for example, be inserted through posterior calcaneal section 106 and into anterior calcaneal section 111. Post 909 and third screw 945 may be, for example, inserted into posterior calcaneal section 106. Fourth screw 946 may be, for example, inserted through anterior calcaneal section 111 and posterior calcaneal section 106 into post hole 925. Thus, implant 900 may have first screw 941 and second screw 943 anchoring implant 900 and posterior calcaneal section 106 to anterior calcaneal section 111; third screw 945 anchoring implant 900 to posterior calcaneal section 106; and fourth screw 946 may, for example, anchor posterior calcaneal section 106 and implant 900 to anterior calcaneal section 111.

Implant installer 200 and shift jig 400 are described for use with implant 300. However, installer 200 and shift jig 400 are similarly compatible for use with implant 600, implant 700, implant 800, and/or implant 900.

Shift jig 480 (FIG. 21) is described for use with implant 300 (FIGS. 5 and 21), engaging post fastener 500 with enclosure wall 321 for post hole 308. However, shift jig 480 is equally compatible with implant 800 (FIG. 24), with a threaded enclosure wall 821 for post hole 808, and implant 900 (FIG. 26), with a threaded enclosure wall 921 for post hole 908. Furthermore, post fastener 500 when engaged with shift jig 480 may be engaged with implant 600 (FIG. 22) by an enclosure (not shown) for third screw hole 604 and implant 700 (FIG. 23) by an enclosure (not shown) for third screw hole 704 or fourth screw hole 706.

In another example, implant installer 200 (FIGS. 3, 4, and 20) and implant 600 (FIG. 22), implant 700 (FIG. 23), implant 800 (FIG. 24), or implant 900 (FIG. 26) may be removably coupled by engaging post fastener 500 (FIGS. 19-20). Post fastener 500 may be inserted through first wire guide hole 203 (as depicted in FIGS. 3, 4, and 20) and threaded section 503 may be threaded into an enclosure (not shown) of third screw hole 604 of implant 600, an enclosure (not shown) for third wire hole 704 or an enclosure (not shown) for fourth wire hole 706 of implant 700, enclosure 821 of post hole 808, or enclosure 921 of post hole 908.

Post fastener 500 is described for use with shift jig 480 however, post fastener 500 (FIGS. 19 and 21) may also be used with shift jig 400 (FIGS. 6 and 12), being insertable through first hole 438 and through coupler 401 for engagement with implant 300, implant 600, implant 700, implant 800, and implant 900. For example, threaded section 503 may be threaded into an enclosure (not shown) of third screw hole 604 of implant 600, an enclosure (not shown) for third wire hole 704 or an enclosure (not shown) for fourth wire hole 706 of implant 700, enclosure 821 of post hole 808, or enclosure 921 of post hole 908.

Embodiments of template 100, implant installer 200, implant 300, implant 600, implant 700, implant 800, implant 900, shift jig 400, shift jig 480, and post fastener 500 may be similarly used for distal bunion osteotomies, where an osteotomy may be, for example, made at a portion of a head of a first metatarsal bone.

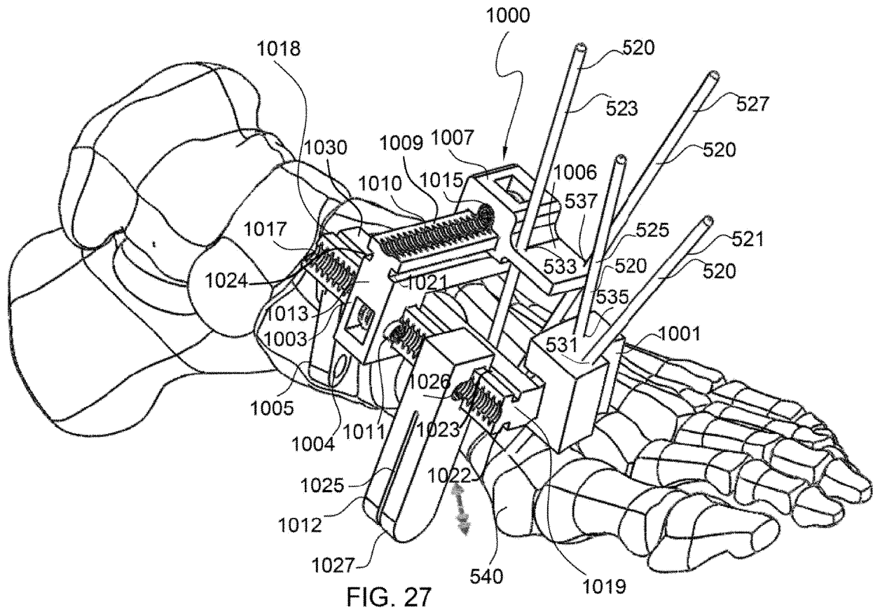

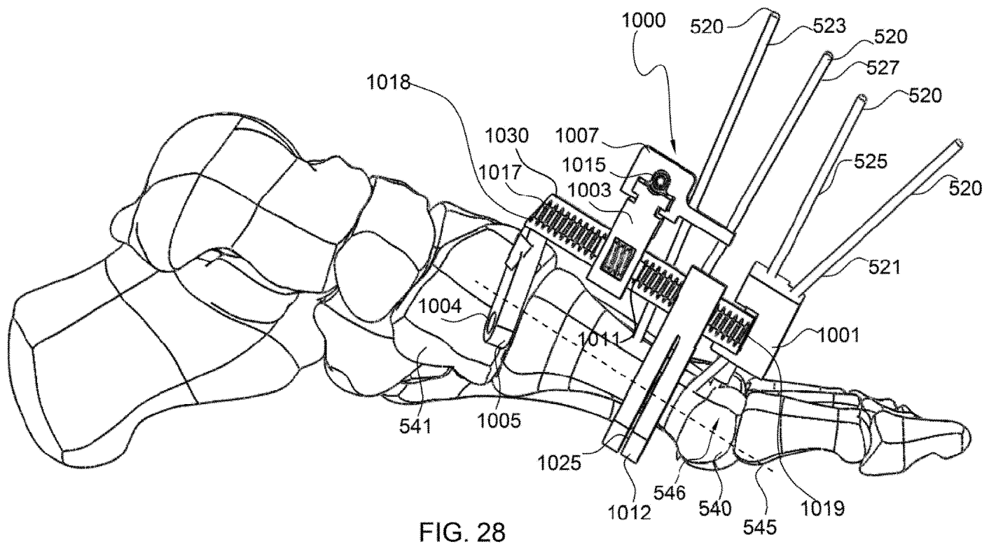

In another example, a shift jig 1000 is depicted in FIGS. 27-34, configured (e.g., shaped and dimensioned) for use in a distal bunion osteotomy. As depicted in FIGS. 27 and 28, shift jig 1000 has a body 1030 including a targeting guide 1005 at a first end 1018 and a coupler 1001 at a second opposite end 1019. A compression-distraction fixture rack 1017 extends longitudinally along an exterior side of body 1030 between first end 1018 and second end 1019. Shift jig 1000 may have, for example, a compression-distraction fixture 1003 housing a first power screw 1011 engaged with compression-distraction fixture rack 1017, with first power screw 1011 acting as a worm gear movably coupled with rack 1017. Compression-distraction fixture 1003 may, for example, further be received in a first recess 1022 and a second recess 1023 of body 1030, movably coupling compression-distraction fixture 1003 to body 1030. First recess 1022 and second recess 1023 may, for example, extend longitudinally along opposite sides of body 1030 between first end 1018 and second end 1019, with first recess 1022 and second recess 1023 positioned on sides of body 1030 adjacent to rack 1017. Compression-distraction fixture 1003 may be, for example, movable along rack 1017 by actuating first power screw 1011 manipulated by a screwdriver (not shown).

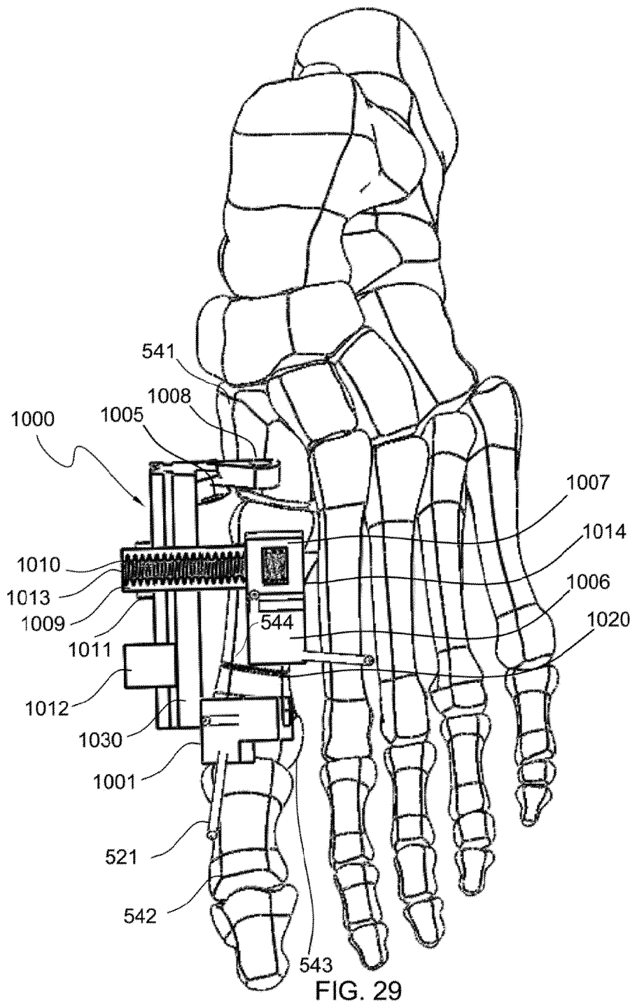

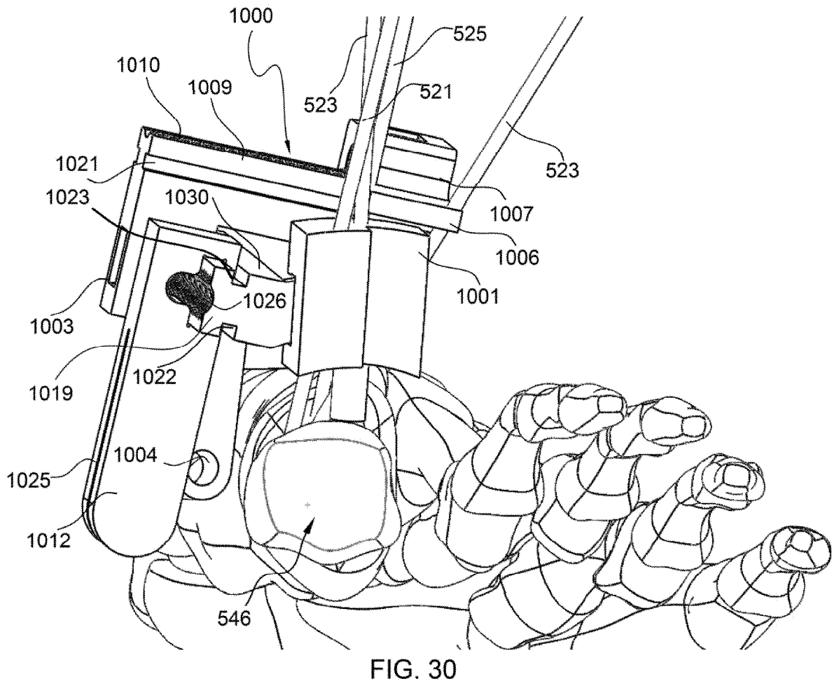

Compression-distraction fixture 1003 may include a guide segment 1009 extending in a direction transverse to body 1030 and having a longitudinal dimension approximately perpendicular to a longitudinal direction of body 1030, as depicted in FIGS. 27 and 29-34. Guide segment 1009 may include a lateral-medial translation rack 1010 extending longitudinally along a top surface between a first end 1013 and a second end 1014 thereof, with first end 1013 and second end 1014 being on opposite sides of body 1030, as depicted in FIGS. 29, 33 and 34. Further, extending in a direction transverse to and approximately perpendicular to a longitudinal direction of body 1030 are a first recess 1021 and a second recess 1024. First recess 1021 and second recess 1023 may, for example, extend longitudinally along opposite sides of guide segment 1009 between first end 1013 and second end 1014, with first recess 1021 and second recess 1024 being on opposite sides of each other and on sides adjacent to rack 1010, as depicted in FIGS. 27, 33 and 34.

With reference to FIGS. 27 and 28, shift jig 1000 may have, for example, a lateral-medial translation fixture 1007 movably coupled with guide segment 1009 and lateral-medial translation rack 1010. Translation fixture 1007 may be engaged with first recess 1021 and second recess 1024. Translation fixture 1007 may house a second power screw 1015. Translation fixture 1007 may be, for example, engaged with rack 1010 and with second power screw 1015 acting as a worm gear movably coupled with rack 1010. Translation fixture 1007 may be, for example, movable along rack 1010 by actuating second power screw 1015 manipulated by a screwdriver (not shown). Translation fixture 1007 may include a guide wire plate 1006 extending away from fixture 1007 in an approximately perpendicular direction to a longitudinal dimension of guide segment 1009.

A cut guide 1012 may be, for example, movably coupled with body 1030 and engaged with first recess 1022 and second recess 1023, as depicted in FIGS. 27, 30, and 32. Cut guide 1012 extends away from body 1030 in an approximately perpendicular direction to the longitudinal dimension of body 1030 and the longitudinal dimension of guide segment 1009. Cut guide 1012 may further have a cut slot 1025 through cut guide 1012, extending from a free end 1027 in the longitudinal dimension towards body 1030 as depicted in FIG. 27. Cut guide 1012 may have a hole 1026 extending therethrough parallel to a longitudinal dimension of body 1030 and adjacent to rack 1017, as depicted in FIGS. 27, 30, and 32, with hole 1026 configured (e.g., shaped and dimensioned) to accommodate a screwdriver (e.g., driver 413 as depicted in FIGS. 10 and 13, or similar) for access to first power screw 1011 of compression-distraction fixture 1003.

Cut slot 1025, as depicted in FIG. 27, may be configured (e.g., shaped and dimensioned) to, for example, accommodate and guide a cutting end of a saw or other medical cutting device (not shown) to make a cut 1020 or similar incision into and/or through a first metatarsal bone 540, as depicted in FIGS. 29, 31, 33, and 34.

In certain embodiments, cut guide 1012 may further house a third power screw (not shown) within hole 1026. Cut guide 1012 may be, for example, engaged with rack 1017 and with the third power screw acting as a worm gear movably coupled with rack 1017. Cut guide 1012 may be, for example, movable along rack 1017 by actuating the third power screw (not shown) manipulated by a screwdriver (not shown).

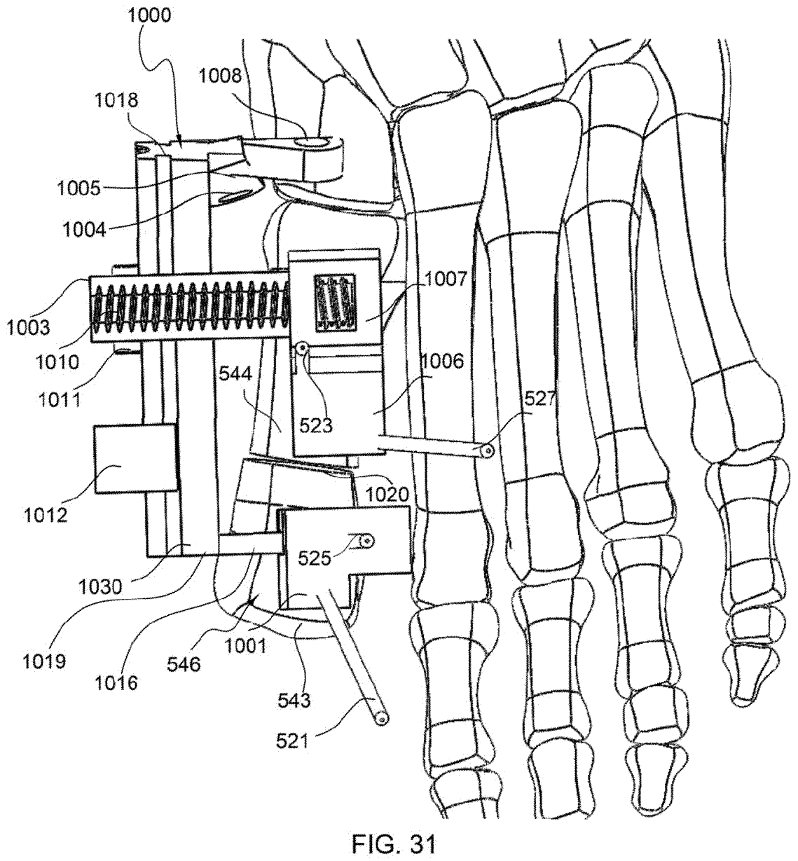

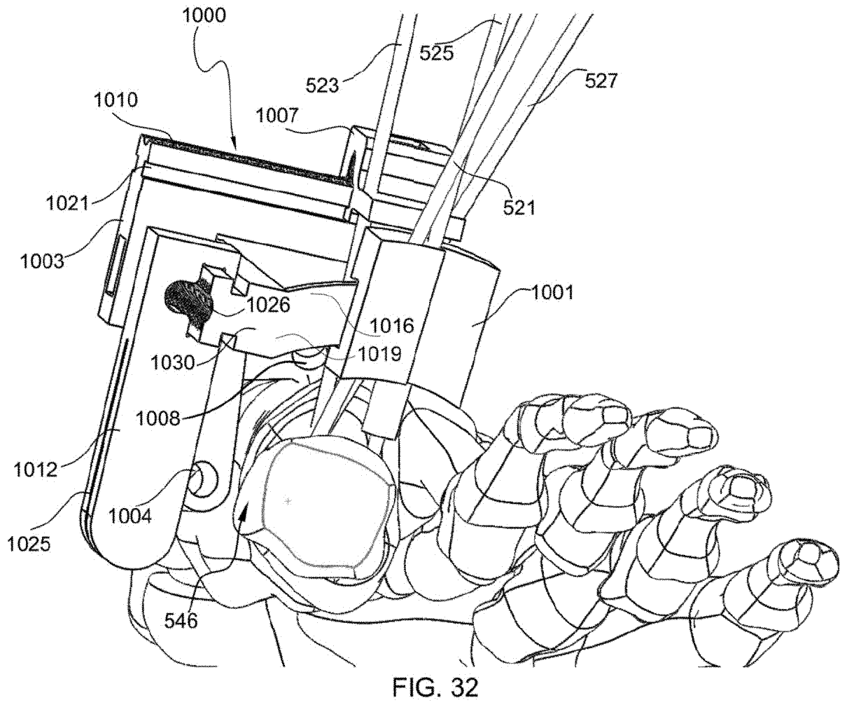

With reference to FIGS. 31-34, a coupler 1001 is movably coupled to an arm 1016 connected to body 1030 at second end 1019, with arm 1016 having a longitudinal dimension extending in an approximately perpendicular direction to a longitudinal dimension of body 1030 and approximately parallel to a longitudinal dimension of guide segment 1009. Coupler 1001 may be, for example, translatable along the longitudinal dimension of arm 1016. Furthermore, arm 1016 may have a curvature along the longitudinal dimension of arm 1016 as depicted in FIG. 32, for example. Coupler 1001 may be moved along an arc of the curvature of arm 1016 as depicted in FIGS. 30 and 32, with arm 1016 having a concave curvature, relative to first metatarsal bone 540, imparting a rotation of a metatarsal anterior section 543, as depicted. Whether shift jig 1000 has arm 1016 with a convex or concave curvature relative to first metatarsal bone 540, with the movement of coupler 1001 approximately imparting an inline rotation of metatarsal anterior section 543 relative to a metatarsal posterior section 544, depends on what is surgically desired. Furthermore, the direction of approximate inline rotation of metatarsal anterior section 543 relative to metatarsal posterior section 544 may depend on the type of defect being corrected.

With reference to FIG. 27, shift jig 1000 may be, for example, removably coupled to first metatarsal bone 540 by guide wires 520 configured (e.g., shaped and dimensioned) for insertion into bone. Guide wires 520 may include, for example, a first guide wire 521, a second guide wire 523, a third guide wire 525, and a fourth guide wire 527. Guide wires may be inserted through wire holes passing through shift jig 1000 and wire holes may include, for example, a first wire hole 531, a second wire hole 533, a third wire hole 535, and a fourth wire hole 537. As depicted in FIG. 27, first wire hole 531 and third wire hole 535 may extend through coupler 1001. Second wire hole 533 and fourth wire hole 537 may extend through guide wire plate 1006. First wire hole 531, second wire hole 533, third wire hole 535, and fourth wire hole 537 may be respectively configured (e.g., shaped and dimensioned) to accommodate first guide wire 521, second guide wire 523, third guide wire 525, and fourth guide wire 527.

Targeting guide 1005 extends away from body 1030 at first end 1018 and has a first targeting aperture 1004 and a second targeting aperture 1008 passing therethrough, as depicted in FIGS. 29 and 31. First targeting aperture 1004 and second targeting aperture 1008 may be angled relative to each other as depicted in FIG. 29. Furthermore, first targeting aperture 1004 and second targeting aperture 1008 may be configured (e.g. shaped and dimensioned) to accommodate a drill guide (e.g., removable drill guide 425 as depicted in FIG. 10) and/or a drill bit (e.g. drill 423 as depicted in FIG. 12) for bone preparation.

With reference to FIGS. 28, 30, and 32, third guide wire 525 may be, for example, centered and inserted into a head region 546 of metatarsal bone 540. Shift jig 1000 may be placed onto third guide wire 525 by inserting third guide wire into third hole 535, positioning shift jig 1000 against a cuneiform 541, first metatarsal 540, and/or a phalange 542 of a foot. Shift jig 1000 is configured (e.g., shaped and dimensioned) for placement onto first metatarsal 540 such that a proposed cut through first metatarsal 540 is positioned between coupler 1001 and wire plate 1006. First guide wire 521, second guide wire 523, and fourth guide wire 527 may be, for example, respectively inserted into first hole 531, second hole 533, and fourth hole 537 and into first metatarsal 540, securing shift jig 1000 to first metatarsal 540. First guide wire 521 and third guide wire 525 may be positioned at nonparallel angles to each other. Second guide wire 523 and fourth guide wire 527 may also be positioned at nonparallel angles to each other.

Cut guide 1012 may be placed onto body 1030 (e.g. engaged with first recess 1022 and second recess 1023 by hand) either before or after shift jig 1000 is secured to first metatarsal 540. With reference to FIG. 29, the position of cut guide 1012 may be adjusted to align with the desired location of a proposed cut to create an osteotomy. Cut guide 1012 may be, for example, positioned longitudinally along body 1030 between translation fixture 1007 and coupler 1001 with first guide wire 521 and third guide wire 525 on one side and second guide wire 523 and fourth guide wire 527 on the other side of cut guide 1012. A position of cut guide 1012 may be adjusted by sliding cut guide 1012 (e.g. by hand manipulation) along the engagement with first recess 1022 and second recess 1023.

With reference to FIG. 28, a blade portion of a medical cutting device (not shown) may be inserted through cut slot 1025 and a cut may be made, as depicted in FIGS. 29 and 31, separating first metatarsal 540 into metatarsal anterior section 543 and a metatarsal posterior section 544. Cut 1020 may be transverse to a longitudinal dimension of first metatarsal 540, such as, for example, a metatarsal axis 545. Metatarsal axis 545 may, for example, run longitudinally through and at an approximate center of first metatarsal 540. Cut guide 1012 may be removed if surgically desired after cut 1020 is made.

With reference to FIGS. 30 and 32, arm 1016 may be curved, and the curvature may be, for example, centered about metatarsal axis 545 (FIG. 28). Moving coupler 1001 along the curvature of arm 1016 may rotate metatarsal anterior section 543, connected to coupler 1001 by first guide wire 521 and third guide wire 525, about metatarsal axis 545 (FIG. 28). By moving coupler 1001 along arm 1016, metatarsal anterior section 543 may be rotated to, for example, correct a frontal plane deformity of first metatarsal 540, with metatarsal anterior section 543 rotated relative to metatarsal posterior section 544. A set screw (not shown) or a thumb tightening element (not shown) may be housed within coupler 1001 and engaged with arm 1016. The set screw (not shown) or thumb tightening element (not shown) may be disengaged from arm 1016 to allow coupler 1001 to move freely along arm 1016 and engaged with arm 1016 to fix the position and orientation of coupler 1001 on arm 1016.

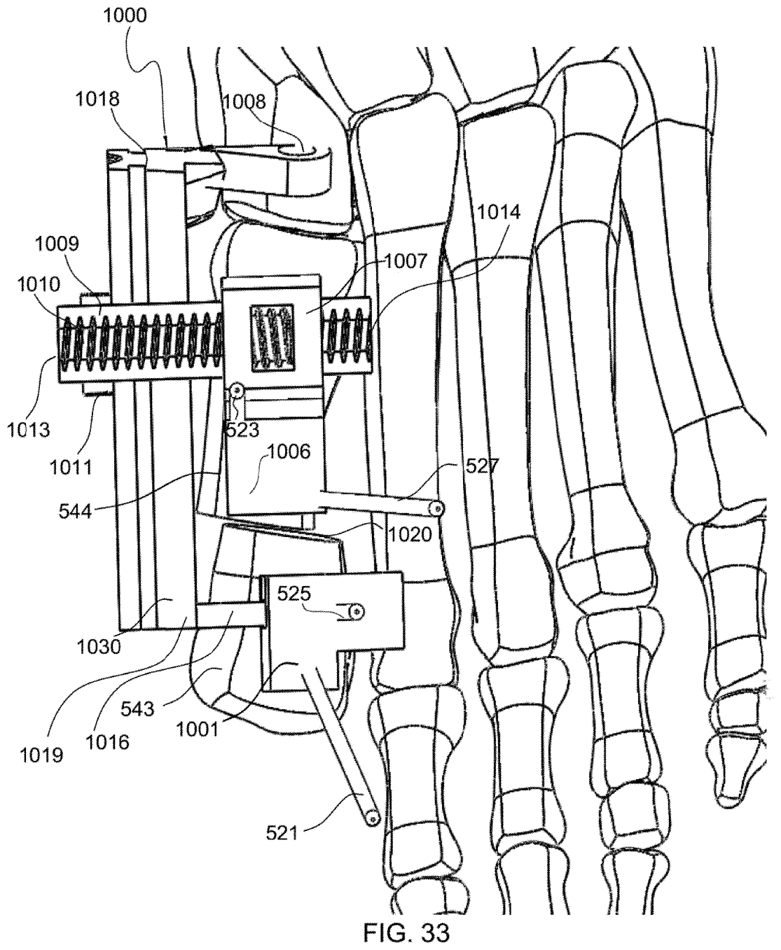

With reference to FIG. 33, second power screw 1015 may be rotated and lateral-medial translation fixture 1007 may be moved along rack 1010. Translation fixture 1007 may be fixed to metatarsal posterior section 544 with guide wires 520 (e.g., second guide wire 523 and fourth guide wire 527), and shift jig 1000 may be translatably moved in a medial-lateral direction relative to translation fixture 1007 and first metatarsal 540, for example. Metatarsal anterior section 543 may be translatably moved, for example, in a lateral direction relative to metatarsal posterior section 544 to a surgically desired position.

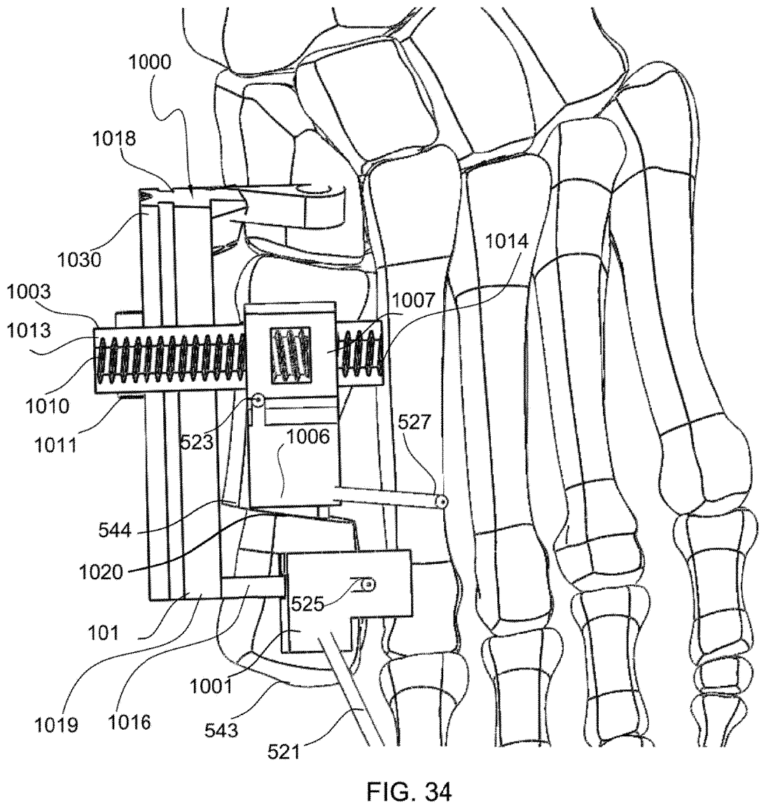

With reference to FIG. 34, first power screw 1011 may be rotated and compression-distraction fixture 1003 may be translatably moved along rack 1017 (FIGS. 27 and 28) to compress or distract metatarsal anterior section 543 and metatarsal posterior section 544.

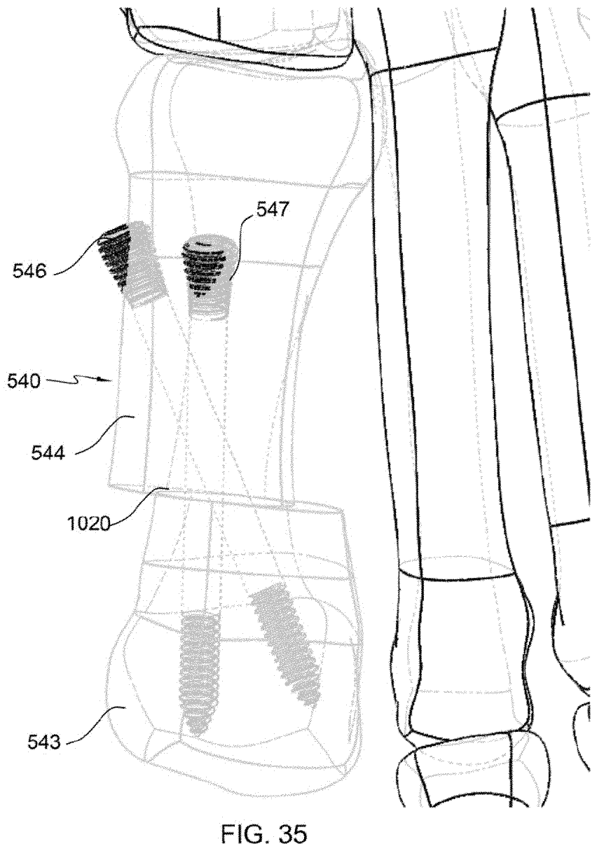

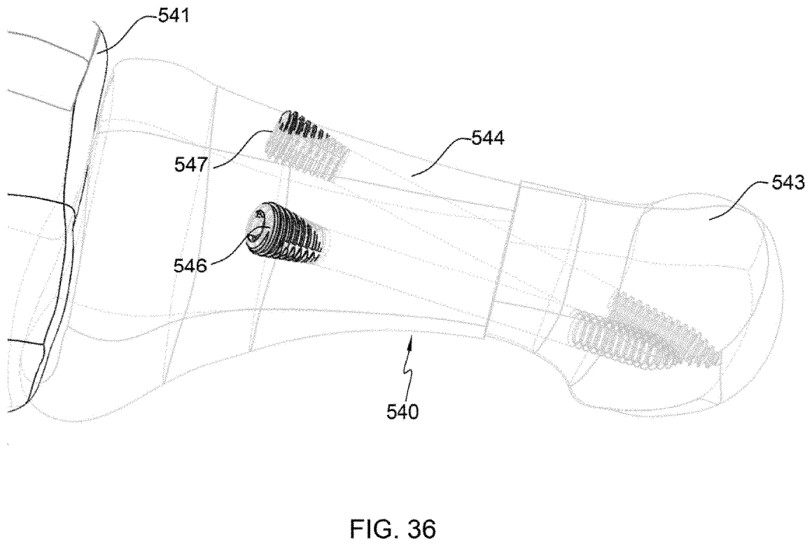

After metatarsal anterior section 543 is placed in a surgically desired position (e.g. via coupler 101, translation fixture 1007, and compression-distraction fixture 1003 described above), first targeting aperture 1004 (FIGS. 31 and 32) and second targeting aperture 1008 (FIGS. 31 and 33) may be used for bone preparation through, for example, drilling. First targeting aperture 1004 and second targeting aperture 1008 may be used to guide screws into and through metatarsal posterior section 544 and metatarsal anterior section 543, as depicted in FIGS. 35 and 36. For example, first screw 546 and second screw 547 may be inserted within first metatarsal 540, connecting metatarsal posterior section 544 and metatarsal anterior section 543. First screw 546 and second screw 547 may be, for example positioned at relative angles to each other. While first screw 546 and second screw 547 are depicted, a single screw or a plurality of screws may be used, as surgically desired. Furthermore, first screw 546 and second screw 547 may be, inserted at nonparallel angles relative to each other for example.

Referring to FIGS. 27-36, a first metatarsal osteotomy procedure is depicted for use on a left foot, the surgical procedure is equally valid on the right foot and shift jig 1000 may be adapted (e.g., shaped and dimensioned) for use with a right foot.

Referring to FIG. 27, an exemplary surgical technique in accordance with the present invention for performing a first metatarsal osteotomy procedure begins by inserting third guide wire 525 into first metatarsal 540 (e.g. metatarsal head region 456 as depicted in FIGS. 30-32), placing shift jig 1000 onto third guide wire 525 by inserting third guide wire 525 into third hole 535, and positioning and seating shift jig 1000 onto first metatarsal 540. The procedure also includes, inserting guide wires 520 through shift jig 1000 with first guide wire 521 inserted into first hole 531, second guide wire 523 inserted into second hole 533, and fourth guide wire 527 inserted into fourth hole 537. Guide wires 520 are inserted at nonparallel angles relative to each other. The order in which guide wires 520 (e.g. first guide wire 521, second guide wire 523, third guide wire 525, and fourth guide wire 527) are inserted into first metatarsal bone 540 and through shift jig 1000 may vary with surgical desire or the bone deformity being corrected. However, guide wires 520 may be placed through shift jig 1000 and into first metatarsal bone 540 to position guide wires 520 on opposite sides of a prospective cut (e.g. cut 1020 of FIG. 29) and to fix shift jig 1000 to the opposite sides of the prospective cut (e.g. cut 1020 of FIG. 29), for example.

Referring to FIGS. 28 and 29, the metatarsal osteotomy procedure further includes positioning cut guide 1012 to align cut slot 1025 with the location of a proposed osteotomy cut, making cut 1020, and separating first metatarsal 540 into metatarsal anterior section 543 and metatarsal posterior section 544. First guide wire 521, second guide wire 523, third guide wire 525, and fourth guide wire 527 may be positioned on opposite sides of cut 1020, with shift jig 1000, for example, fixed via first guide wire 521 and third guide wire 525 to metatarsal anterior section 543, and second guide wire 523 and fourth guide wire 527 to metatarsal posterior section 544.

Referring to FIGS. 30 and 32, the procedure includes moving coupler 1001 to rotate metatarsal anterior section 543 around metatarsal axis 545 (FIG. 28) relative to metatarsal posterior section 544.

In another example, second power screw 1015 may be rotated to translatably move lateral-medial fixture 1007 along rack 1010, and move metatarsal anterior section 543 laterally relative to metatarsal posterior section 544 as surgically desired and as depicted in FIG. 33. First power screw 1011, as described above, may be rotated to move compression-distraction fixture 1003 along rack 1017 to compress or distract metatarsal anterior section 543 relative to metatarsal posterior section 544, as surgically desired and as depicted in FIG. 34.

After metatarsal anterior section 543 is positioned relative to metatarsal posterior section 544, as described above, screws may be inserted to fix metatarsal posterior section 544 and metatarsal anterior section 543, as depicted in FIGS. 35 and 36. In one example, first screw 546 and second screw 547 may be inserted through metatarsal posterior section 544 and into metatarsal anterior section 543 at nonparallel angles relative to each other.

Shift jig 1000 may be configured (e.g., shaped, dimensioned, or otherwise arranged) for use with calcaneal osteotomies. Shift jig 400, shift jig 480, and shift jig 1000 are described for use with calcaneal osteotomies and/or distal bunion osteotomies; however, the devices and procedures may be configured (e.g., shaped, dimensioned, and otherwise arranged) for use with bone osteotomy procedure that involves compression/distraction and lateral/medial positioning and fixation. Furthermore, shift jig 400, shift jig 480, and shift jig 1000 may be configured (e.g., shaped, dimensioned, and otherwise arranged) for use in bone osteotomy procedures that involve compression/distraction and dorsal/plantar positioning and fixation. Still further, shift jig 400, shift jig 480, and shift jig 1000 may be configured (e.g., shaped, dimensioned, and otherwise arranged) for use in bone osteotomy procedures that involve compression/distraction and any relative transverse bone positioning and fixation.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

We claim:

1. A bone displacement system comprising:
a body having a first arm and a second arm;
said first arm coupled to a first bone contacting leg configured to be connected to a first bone portion;
said second arm coupled to a second bone contacting leg configured to be connected to a second bone portion;
a force application fixture coupled to said first arm and said first bone contacting leg and configured to apply a force between said first arm and said first bone contacting leg.

2. The system of claim 1 wherein the applying the force by said force application fixture between said first arm and said first bone contacting leg comprises applying the force to compress said first bone and said second bone portion toward each other.

3. The system of claim 1 wherein the applying the force by said force application fixture between said first arm and said first bone contacting leg comprises applying the force to distract said first bone relative to said second bone portion relative to each other.

4. The system of claim 1 wherein the applying the force by said force application fixture between said first arm and said first bone contacting leg comprises applying the force to displace said first bone relative to said second bone portion in a medial-lateral direction.

5. The system of claim 1 wherein said first arm is movable relative to said first bone contacting leg portion such that the applying the force by said force application fixture between said first arm and said first bone contacting leg causes said first bone portion to move in a first direction relative to said second bone portion when said first arm is coupled to said first bone portion and said arm is coupled to said second bone portion.

6. The system of claim 5 wherein the first direction is a medial-lateral direction perpendicular to a longitudinal axis of said first arm.

7. The system of claim 5 wherein the first direction is an anterior-posterior direction parallel to a longitudinal axis of said first arm.

8. The system of claim 1 wherein said force application fixture is configured to allow a user to apply the force between said first arm and said first bone contacting leg to cause the force to be applied between said first bone portion and said second bone portion.

9. The system of claim 1 wherein said first arm comprises a rack engageable with a screw of said force application fixture to allow a user to turn the screw to apply the force.

10. The system of claim 1 wherein said first bone contacting leg is connected to said first arm and extends downwardly toward said first bone portion and said second bone contacting leg is connected to said second arm and extends downwardly toward said second bone portion.

11. The system of claim 1 further comprising a second force application fixture configured to apply a second force between said body and said second bone contacting leg.

12. The system of claim 11 wherein said second bone contacting leg comprises a rack engageable with a screw of said second force application fixture to allow a user to turn the screw to apply the force, said second force application connected to said first arm.

13. The system of claim 1 wherein said bone contacting leg comprises a coupler connected to said body and an implant engaged with said coupler, said implant configured to be connected to the first bone portion.

14. The system of claim 1 wherein said first arm has a longitudinal axis perpendicular to a second longitudinal axis of said second arm.

15. A method for use in displacing a bone, comprising:
coupling a first arm of a body to a first bone portion via a first bone contacting leg connected to the body;
coupling a second arm of the body to a second bone portion via a second bone contacting leg connected to the body; and
applying a force between the first arm and the first bone contacting leg via a force application fixture coupled to the first arm and the first bone contacting leg.

16. The method of claim 15 wherein the applying the force by the force application fixture between the first arm and the first bone contacting leg comprises applying the force to compress the first bone and the second bone portion toward each other.

17. The method of claim 15 wherein the applying the force by the force application fixture between the first arm and the first bone contacting leg comprises applying the force to distract the first bone relative to the second bone portion relative to each other.

18. The method of claim 15 wherein the applying the force by the force application fixture between the first arm and the first bone contacting leg comprises applying the force to displace the first bone relative to the second bone portion in a medial-lateral direction.

19. The method of claim 15 wherein the first arm is movable relative to the first bone contacting leg portion such that the applying the force by the force application fixture between the first arm and the first bone contacting leg causes the first bone portion to move in a first direction relative to the second bone portion when the first arm is coupled to the first bone portion and the arm is coupled to the second bone portion.

20. The method of claim 19 wherein the first direction is a medial-lateral direction perpendicular to a longitudinal axis of the first arm.

21. The method of claim 19 wherein the first direction is an anterior-posterior direction parallel to a longitudinal axis of the first arm.

22. The method of claim 15 wherein the applying the force comprises a user causing the force application fixture to apply the force.

23. The method of claim 22 wherein the applying the force comprises the user turning a screw of the force application fixture engaged with a rack of the first arm to apply the force.

24. The method of claim 15 further comprising applying a second force between the body and the second bone contacting leg by a user using a second force application fixture coupled to the body and the second bone contacting leg.

25. The method of claim 24 wherein the applying the second force by the user comprises the user turning a screw of the second force application fixture engaged with a rack of the second bone contacting leg and the applying the force comprises the user turning a screw of the force application fixture engaged with a rack of the first arm to apply the force.

26. The method of claim 25 wherein the applying the second force causes a movement of the first bone portion relative to the second bone portion.

27. The method of claim 25 wherein the force and the second force are perpendicular to each other.

28. The method of claim 25 wherein the applying the force between the first arm and the first bone contacting leg via the force application fixture coupled to the first arm and the first bone contacting leg comprises applying the force to compress the first bone and the second bone portion toward each other.

29. The method of claim 15 wherein the bone contacting leg comprises a coupler connected to the body and an implant engaged with said coupler, and further comprising connecting the implant to the first bone portion.
